(19) United States
(12) Patent Application Publication
(10) Pub. No.: US 2026/0225981 A1
(43) Pub. Date: Aug. 6, 2026

(54) PROCESS FOR PREPARING ALLYLIC ALCOHOL

(71) Applicant: Firmenich SA, Satigny (CH)

(72) Inventors: Lucia BONOMO, Satigny (CH); Denis JACOBY, Satigny (CH); Pascal ARBICHA, Dax (FR); Fabien FONTENY, Satigny (CH); Fabrice KELLER, Satigny (CH)

(21) Appl. No.: 19/137,589

(22) PCT Filed: Dec. 15, 2023
(86) PCT No.: PCT/EP2023/086048

§ 371 Date: Jun. 10, 2025

(30) Foreign Application Priority Data
Dec. 15, 2022 (EP) 22213684.8

(51) Int. Cl. C07C 29/56; B01J 31/22

(52) U.S. Cl. CPC C07C 29/56; B01J 31/2243; B01J 2231/52; B01J 2531/26

(57) ABSTRACT

Disclosed herein is a process for the rearrangement of epoxides into allylic alcohols in a presence of zinc catalyst and two amino phenol.

### TECHNICAL FIELD

The present invention relates to the field of organic synthesis and more specifically it concerns a process for the rearrangement of epoxides into allylic alcohols in a presence of zinc catalyst and amino phenol.

### BACKGROUND

Allylic alcohols represent skeletons highly desirables which could be used as such or as key intermediates useful to prepare more complex compounds in different fields such as, among others, perfumery, cosmetic, pharmaceutic or agrochemistry. In particular, carveol is a valuable compound known as perfuming ingredient or could be a key intermediate toward more complex compounds. Carveol may be obtained via the rearrangement of the limonene oxide prepared from limonene, i.e. a renewable carbon source, preventing the use of petrochemical starting material. Said access is particular sought today wherein the demand of products with green properties, low environmental and social impact are increasing. The rearrangement of an epoxide into an allylic alcohol has been disclosed in US2003065230 and WO2021151790 wherein such rearrangement is performed in the presence of a zinc catalyst and an activator. However, the reported conditions request a temperature of at least 200° C. to reach complete conversions and good yields. Such a high temperature may be difficult to implement industrially and seek a high amount of energy. In addition, US2003065230 and WO2021151790 report the one-pot rearrangement of epoxide followed by an oxidation in order to obtain an enone. The oxidation is performed in the presence of the hydrogen acceptor, even called a sacrificial agent. Said hydrogen acceptor is added in increments and, then, the alcohol formed and non-reacted hydrogen acceptor are removed after each increment. The hydrogen acceptor or the corresponding alcohol is not recovered or recycled at the end of the process.

So, today there is a need to develop sustainable process while reducing the energy consumption and the generation of waste.

The present invention allows the rearrangement of an epoxide into an allylic alcohol under catalytic conditions and at a lower temperature without compromise on yield while reducing side product formation. On top of that, the invention's conditions allow to perform in one-pot the rearrangement of an epoxide into an allylic alcohol under catalytic conditions in the presence of hydrogen acceptor followed by an oxidation under catalytic conditions providing the desired compound in high yield while limiting the generation of waste and even allowing the recycling of the hydrogen acceptor or the corresponding alcohol. To the best of our knowledge, none of the reported techniques are capable to combine these two steps to one-pot produce the corresponding alpha-beta unsaturated compound in high yields and in a sustainable manner.

### SUMMARY OF THE INVENTION

The invention relates to a novel process allowing the preparation of allylic alcohol using starting material comprising renewable carbon under mild conditions never reported or suggested in the prior art.

So, a first object of the present invention is a process for the rearrangement of an epoxide into an allylic alcohol wherein said process is carried out in the presence of
i) a catalyst of formula Zn(aa)₂ wherein aa is an α-amino carboxylate having at least 3 carbon atoms or a β-amino carboxylate; and
ii) an amino phenol.

Another object of the inventions is a process for the rearrangement of epoxides into allylic alcohols wherein said process is carried out in the presence of
i) a catalyst of formula Zn(carboxylate)₂; and
ii) an amino phenol;
   wherein the mole ratio between the catalyst formula Zn(carboxylate)₂ and the amino phenol is comprised between 1:3 and 1:5.

A further object of the inventions is a catalytic system comprising or consisting of
i) a catalyst of formula Zn(aa)₂ wherein aa is an α-amino acid carboxylate having at least 3 carbon atoms or a β-amino carboxylate; and
ii) an amino phenol.

### DESCRIPTION OF THE INVENTION

Surprisingly, it has now been discovered that an allylic alcohol could be prepared by the rearrangement of the epoxide an advantageous manner by means of zinc catalyst and in the presence of amino phenol. These unprecedented conditions allow the generation of an alpha, beta-unsaturated carbonyl compound in very high yield without requesting a high temperature. In addition, the invention's conditions may be used in one step process wherein the rearrangement of the epoxide into an allylic alcohol is followed by an oxidation in presence of a zirconium catalyst and a hydrogen acceptor. The one-pot process may be performed at a lower temperature allowing a broader scope of hydrogen acceptor while avoiding the generation of side products and allowing to recover the excess of the hydrogen acceptor and the reduced product; i.e. the corresponding alcohol.

Therefore, a first object of the present invention is a process for the rearrangement of an epoxide into an allylic alcohol wherein said process is carried out in the presence of
iii) a catalyst of formula Zn(aa)₂ wherein aa is an α-amino carboxylate having at least 3 carbon atoms or α-amino carboxylate; and
iv) an amino phenol.

For the sake of clarity, by the expression “α-amino carboxylate having at least 3 carbon atoms”, or the similar, it is meant the normal meaning understood by a person skilled in the art, i.e. a compound formula R^{a}R^{b}C(NHR^{c})COO⁻ wherein at least one group among R^{a}, R^{b} and R^{c} comprises at least one carbon atom. In other words, the α-amino carboxylate is a carboxylate wherein the carbon in alpha of the carboxylate is substituted by an amine functional group provided that the α-amino carboxylate is not glycinate; i.e. CH₂(NH₂)COO⁻. The α-amino carboxylate is a α-amino acid carboxylate. It is understood that orotate is not encompassed by the term “α-amino carboxylate”.

For the sake of clarity, by the expression “β-amino carboxylate”, or the similar, it is meant the normal meaning understood by a person skilled in the art, i.e. a carboxylate wherein the carbon in beta of the carboxylate is substituted by an amine functional group.

According to any embodiments of the invention, the epoxide is a compound of formula

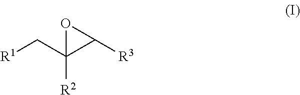

in a form of any one of its stereoisomers or a mixture thereof and wherein and wherein R¹ is a hydrogen atom or a C₁₋₁₀ alkyl group or a C₂₋₁₀ alkenyl, R² is a hydrogen atom or a C₁₋₃ alkyl group, R³ is a hydrogen atom or a C₁₋₅ alkyl group; or R¹ and R³ are taken together and represent a C₂₋₁₆ alkanediyl or a C₃₋₁₆ alkenediyl group; or R² and R³ are taken together and represent a C₂₋₁₆ alkanediyl group; or R¹ and R² are taken together and represent a C₂₋₁₆ alkanediyl or a C₃₋₁₆ alkenediyl group.

According to any embodiments of the invention, the epoxide is a compound of formula

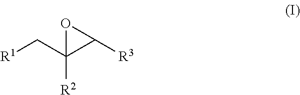

in a form of any one of its stereoisomers or a mixture thereof and wherein and wherein R¹ is a C₁₋₁₀ alkyl group, R² is a hydrogen atom or a C₁₋₃ alkyl group, R³ is a C₁₋₃ alkyl group; or R¹ and R³ are taken together and represent a C₂₋₁₆ alkanediyl or group; or R² and R³ are taken together and represent a C₂₋₁₆ alkanediyl group; or R¹ and R² are taken together and represent a C₂₋₁₆ alkanediyl group.

According to any embodiments of the invention, the allylic alcohol is a compound of formula

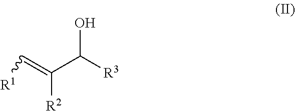

in a form of any one of its stereoisomers or a mixture thereof and wherein R¹, R² and R³ have the same meaning as defined above.

For the sake of clarity, by the expression “any one of its stereoisomers or a mixture thereof”, or the similar, it is meant the normal meaning understood by a person skilled in the art, i.e. that the compounds cited in the invention can be a pure enantiomer or a mixture of enantiomers. In other words, the compounds cited in the invention may possess at least one stereocenter which can have two different stereochemistries (e.g. R or S), e.g. the R¹ group may comprise at least one stereocenter. Said compounds may even be in the form of a pure enantiomer or in the form of a mixture of enantiomers. The compounds cited in the invention may even be in the form of a pure diastereoisomer or in the form of a mixture of diastereoisomer when said compounds possess more than one stereocenter. Said compounds can be in a racemic form or scalemic form. Therefore, said compounds can be one stereoisomer or in the form of a composition of matter comprising, or consisting of, various stereoisomers.

The wavy line indicates that the double bond may be in the form of its E or Z isomer or of a mixture thereof; e.g. the invention comprises compositions of matter consisting of one or more compounds of formula (II) having the same chemical structure but differing by the configuration of the double bond.

According to any one of the above embodiments of the invention, the compound of formula (II) can be in the form of its E or Z isomer or of a mixture thereof, e.g. the invention comprises compositions of matter consisting of one or more compounds of formula (II), having the same chemical structure but differing by the configuration of the doubles bond.

The term “alkyl group “alkenyl group” or “alkoxy group” are understood as comprising linear or branched alkyl, alkenyl or alkoxy groups. The term “alkanediyl group” or “alkenediyl group” are understood as comprising linear, branched, alicylic or cyclic alkanediyl or alkenediyl groups. The terms “alkenyl”; “alkenediyl” and “cycloalkenyl” are understood as comprising 1, 2 or 3 olefinic double bonds, preferably 1 or 2 olefinic double bonds, even more preferably 1 double bond. The term “cycloalkenyl” is understood as comprising a monocyclic or fused, spiro and/or bridged bicyclic or tricyclic cycloalkenyl group, preferably monocyclic cycloalkenyl group.

According to any one of the above embodiments, R¹ may be a hydrogen atom or a C₁₋₈ alkyl or a C₂₋₈ alkenyl group. Particularly, R¹ may be a hydrogen atom or a C₁₋₆ alkyl or a C₂₋₆ alkenyl group. Particularly, R¹ may be a hydrogen atom or a C₁₋₄ alkyl or C₄₋₆ alkenyl group. Particularly, R¹ may be a hydrogen atom or a C₁₋₃ alkyl or C₄₋₆ alkenyl group. Particularly, R¹ may be a hydrogen atom or a C₁₋₃ alkyl or C₄₋₆ alkenyl group. Particularly, R¹ may be a hydrogen atom or a C₁₋₂ alkyl or C₆ alkenyl group. Even more particularly, R¹ may be a hydrogen atom or a methyl, ethyl, hex-3en-1-yl or a 4-methylpent-3-en-1-yl.

According to any one of the above embodiments, R³ may be a hydrogen atom or a C₁₋₅ alkyl group. Particularly, R³ may be a hydrogen atom, a C₁₋₃ alkyl group or a pentyl group. Particularly, R³ may be a hydrogen atom or a methyl, ethyl or pentyl group. Particularly, R³ may be a hydrogen atom or a pentyl group. Even more particularly, R³ may be a hydrogen atom or a linear pentyl group.

According to any one of the above embodiments, R² may be a hydrogen atom or a C₁₋₂ alkyl group. Particularly, R² may be a hydrogen atom or a methyl group. Even more particularly, R² may be a methyl group.

According to any one of the above embodiments, R¹ and R² are taken together and represent a C₂₋₁₂ alkanediyl or C₃₋₁₂ alkenediyl group. Particularly, R¹ and R² are taken together and represent a C₂₋₁₀ alkanediyl or C₃₋₁₀ alkenediyl group. Particularly, R¹ and R² are taken together and represent a C₂₋₈ alkanediyl or C₃₋₈ alkenediyl group. Particularly, R¹ and R² are taken together and represent a C₄₋₈ alkanediyl or C₄₋₈ alkenediyl group. Particularly, R¹ and R² are taken together and form a C₅₋₆ cycloalkenyl optionally substituted by a C₁₋₄ alkyl or alkenyl group. Even more particularly, R¹ and R² are taken together and represent a 2-isopropenyl-1,4-butanediyl or a group of formula (a)

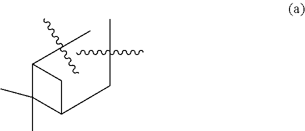 [SMILES: *CC1CC(*)C1(C)C]

in a form of any one of its stereoisomers or a mixture thereof.

According to any one of the above embodiments, R² and R³ are taken together and represent a C₂₋₁₂ alkanediyl group. Particularly, R² and R³ are taken together and represent a C₂₋₁₀ alkanediyl group. Particularly, R² and R³ are taken together and represent a C₂₋₈ alkanediyl group. Particularly, R² and R³ are taken together and represent a C₄₋₈ alkanediyl group. Particularly, R² and R³ are taken together and represent a C₆₋₈ alkanediyl group. Even more particularly, R² and R³ are taken together and represent a group of formula (a)

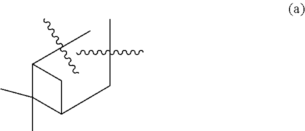 [SMILES: *CC1CC(*)C1(C)C]

in a form of any one of its stereoisomers or a mixture thereof.

According to any one of the above embodiments, R¹ and R³ are taken together and represent a C₂₋₁₂ alkanediyl or C₃₋₁₂ alkenediyl group. Particularly, R¹ and R³ are taken together and represent a C₂₋₁₀ alkanediyl or C₃₋₁₀ alkenediyl group. Particularly, R¹ and R³ are taken together and represent a C₂₋₈ alkanediyl or C₃₋₈ alkenediyl group. Particularly, R¹ and R³ are taken together and represent a C₄₋₈ alkanediyl or C₄₋₈ alkenediyl group. Particularly, R¹ and R³ are taken together and represent a C₅₋₈ alkanediyl or C₅₋₈ alkenediyl group. Particularly, R¹ and R³ are taken together and represent a C₅₋₇ alkanediyl or C₅₋₇ alkenediyl group. Particularly, R¹ and R³ are taken together and represent a C₆ alkanediyl or C₆ alkenediyl group. Even more particularly, R¹ and R³ are taken together and represent a 2-isopropenyl-1,3-propanediyl group, a group of formula (b) or a group of formula (c)

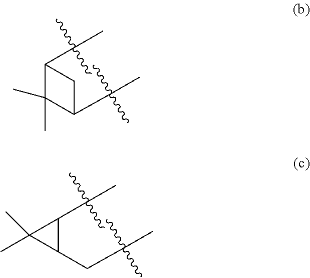

in a form of any one of their stereoisomers or a mixture thereof.

According to any one of the above embodiments, R¹ and R³ are taken together and represent a C₂₋₁₂ alkanediyl or C₃₋₁₄ alkenediyl group and R² is a hydrogen atom or a C₁₋₃ alkyl group. Particularly, R¹ and R³ are taken together and represent a C₂₋₈ alkanediyl or C₄₋₁₂ alkenediyl group and R² is a hydrogen atom or a C₁₋₃ alkyl group. Particularly, R¹ and R³ are taken together and represent a C₂₋₆ alkanediyl or C₄₋₁₀ alkenediyl group and R² is a hydrogen atom or a C₁₋₃ alkyl group. Particularly, R¹ and R³ are taken together and represent a C₂₋₄ alkanediyl or C₄₋₈ alkenediyl group and R² is a hydrogen atom or a C₁₋₃ alkyl group. Particularly, R¹ and R³ are taken together and represent a C₂₋₄ alkanediyl or C₄₋₈ alkenediyl group and R² is a hydrogen atom or a C₁₋₂ alkyl group. Particularly, R¹ and R³ are taken together and represent a C₂₋₃ alkanediyl or C₅₋₇ alkenediyl group and R² is a hydrogen atom or a C₁₋₂ alkyl group. Even more particularly, R¹ and R³ are taken together and represent a C₃ alkanediyl or C₆ alkenediyl group and R² is a methyl group.

According to any one of the above embodiments of the invention, non-limiting examples of the epoxide are 1,2-limonene oxide, 3,8,8-trimethyl-4-oxatricyclo[5.1.0.0^{3,5}]octane, 4,8,8-trimethyltricyclo[5.1.0.0^{2,4}]octane, 2-isopropyl-5-methyl-7-oxabicyclo[4.1.0]heptane, 4-isopropyl-1-methyl-7-oxabicyclo[4.1.0]heptane, alpha-pinene oxide, beta-pinene oxide, 2,2,6-trimethyl-1-oxaspiro[2.5]oct-5-ene, 2,2,6-trimethylspiro[2.5]oct-4-ene and caryophyllene oxide.

According to any one of the above embodiments of the invention, non-limiting examples of the allylic alcohol are carveol, 4,7,7-trimethylbicyclo[4.1.0]hept-4-en-3-ol, 3,7,7-trimethylbicyclo[4.1.0]hept-3-en-2-ol, 6-isopropyl-3-methylcyclohex-2-en-1-ol, 5-isopropyl-2-methylcyclohex-2-en-1-ol, 6,6-dimethyl-2-methylenebicyclo[3.1.1]heptan-3-ol, (6,6-dimethylbicyclo[3.1.1]hept-2-en-2-yl)methanol.

According to any embodiments of the invention, the α-amino carboxylate has at least 4 carbon atoms, preferably at least 5 carbon atom, even more preferably at least 6 carbon atom.

According to any embodiments of the invention, the α-amino carboxylate is a compound of formula R^{a}R^{b}C(NHR^{c})COO⁻ wherein R^{a} is a C₁₋₁₈ hydrocarbon optionally comprising one or two functional groups selected amongst ether, ester, carbonyl, amine, amide, alcohol, hydroxy, thioether, carboxylic acid groups, R^{b}, is a hydrogen atom or a C₁₋₁₈ hydrocarbon optionally comprising one or two functional groups selected amongst ether, ester, carbonyl, amine, amide, alcohol, hydroxy, thioether, carboxylic acid groups, R^{c} is a hydrogen atom or a C₁₋₁₈ hydrocarbon optionally comprising one or two functional groups selected amongst ether, ester, carbonyl, amine, amide, alcohol, hydroxy, thioether, carboxylic acid groups; or R^{a} and R^{c}, when taken together, represent a C₃₋₁₁ alkanediyl group optionally comprising one or two functional groups selected amongst ether, ester, carbonyl, amine, amide or alcohol groups. When Re is a C₁₋₁₈ hydrocarbon comprising one or two carbonyl functional groups, then the carbonyl functional group is not directly connected to the NH.

It is understood that by “ . . . hydrocarbon group . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynyl group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

It is understood that with the terms “ . . . a hydrocarbon group, optionally comprising . . . ”, it is meant that said hydrocarbon group optionally comprises alcohol, ketone, aldehyde, ether, ester, carboxylic acid, amine, amide, carbamate or nitrile. These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain. For example, a —CH₂—CH₂—CHOH—CH₂— group represents a C₄ hydrocarbon group comprising an alcohol group (substitution of a hydrogen atom) and, similarly, a —CH₂—CH₂—O—CH₂—CH₂—O—CH₂—CH₂— group represents a C₆ hydrocarbon group comprising two ether groups (substitution of carbon atoms/insertion into the hydrocarbon chain).

The term “optionally” is understood that a group can or cannot comprise a certain functional group or substituent.

According to any embodiments of the invention, R^{a} may be a C₁₋₁₅ hydrocarbon optionally comprising one or two functional groups selected amongst ether, ester, carbonyl, amine, amide, alcohol, hydroxy, thioether, carboxylic acid groups. Particularly, R^{a} may be a C₁₋₁₂ hydrocarbon group optionally comprising one or two functional groups selected amongst ether, ester, carbonyl, amine, amide, alcohol, hydroxy, thioether, carboxylic acid groups. Particularly, R^{a} may be a C₁₋₁₀ hydrocarbon group optionally comprising one or two functional groups selected amongst ether, ester, carbonyl, amine, amide, alcohol, hydroxy, thioether, carboxylic acid groups. Particularly, R^{a} may be a C₁₋₈ hydrocarbon group optionally comprising one or two functional groups selected amongst ether, ester, carbonyl, amine, amide, alcohol, hydroxy, thioether, carboxylic acid groups. Particularly, R^{a} may be a phenyl group, a benzyl group or a C₁₋₈ alkyl group optionally comprising one or two functional groups selected amongst ether, ester, carbonyl, amine, amide, alcohol, hydroxy, thioether, carboxylic acid groups. Even more particularly, R^{a} may be a phenyl group, a benzyl group or a C₁₋₆ alkyl group optionally comprising one or two functional groups selected amongst ether, ester, carbonyl, amine, amide, alcohol, hydroxy, thioether, carboxylic acid groups.

According to any embodiments of the invention, R^{b} may be a hydrogen atom or a C₁₋₁₅ hydrocarbon optionally comprising one or two functional groups selected amongst ether, ester, carbonyl, amine, amide, alcohol, hydroxy, thioether, carboxylic acid groups. Particularly, R^{b} may be a hydrogen atom or a C₁₋₁₂ hydrocarbon group optionally comprising one or two functional groups selected amongst ether, ester, carbonyl, amine, amide, alcohol, hydroxy, thioether, carboxylic acid groups. Particularly, R^{b} may be a hydrogen atom or a C₁₋₁₀ hydrocarbon group optionally comprising one or two functional groups selected amongst ether, ester, carbonyl, amine, amide, alcohol, hydroxy, thioether, carboxylic acid groups. Particularly, R^{b} may be a hydrogen atom or be a C₁₋₈ hydrocarbon group optionally comprising one or two functional groups selected amongst ether, ester, carbonyl, amine, amide, alcohol, hydroxy, thioether, carboxylic acid groups. Particularly, R^{b} may be a hydrogen atom or be a C₁₋₈ alkyl group optionally comprising one or two functional groups selected amongst ether, ester, carbonyl, amine, amide, alcohol, hydroxy, thioether, carboxylic acid groups. Particularly, R^{b} may be a hydrogen atom or be a C₁₋₆ alkyl group. Particularly, R^{b} may be a hydrogen atom or be a C₁₋₄ alkyl group. Particularly, R^{b} may be a hydrogen atom or be a C₁₋₃ alkyl group. Particularly, R^{b} may be a hydrogen atom or be a C₁₋₂ alkyl group. Particularly, R^{b} may be a hydrogen atom or a methyl group. Even more particularly, R^{b} may be a hydrogen atom.

According to any embodiments of the invention, Re may be a hydrogen atom or a C₁₋₁₅ hydrocarbon optionally comprising one or two functional groups selected amongst ether, ester, carbonyl, amine, amide, alcohol, hydroxy, thioether, carboxylic acid groups. Particularly, R^{c} may be a hydrogen atom or a C₁₋₁₂ hydrocarbon group optionally comprising one or two functional groups selected amongst ether, ester, carbonyl, amine, amide, alcohol, hydroxy, thioether, carboxylic acid groups. Particularly, Re may be a hydrogen atom or a C₁₋₁₀ hydrocarbon group optionally comprising one or two functional groups selected amongst ether, ester, carbonyl, amine, amide, alcohol, hydroxy, thioether, carboxylic acid groups. Particularly, Re may a hydrogen atom or be a C₁₋₈ hydrocarbon group optionally comprising one or two functional groups selected amongst ether, ester, carbonyl, amine, amide, alcohol, hydroxy, thioether, carboxylic acid groups. Particularly R^{c} may be a hydrogen atom, a phenyl group, a benzyl group or a C₁₋₈ alkyl group optionally comprising one or two functional groups selected amongst ether, ester, carbonyl, amine, amide, alcohol, hydroxy, thioether, carboxylic acid groups. Particularly, R^{c} may a hydrogen atom or be a C₁₋₈ alkyl group optionally comprising one or two functional groups selected amongst ether, ester, carbonyl, amine, amide, alcohol, hydroxy, thioether, carboxylic acid groups. Particularly, R^{c} may a hydrogen atom or be a C₁₋₆ alkyl group. Particularly, Re may a hydrogen atom or be a C₁₋₄ alkyl group. Particularly, Re may a hydrogen atom or be a C₁₋₃ alkyl group. Particularly, Re may a hydrogen atom or be a C₁₋₂ alkyl group. Particularly, Re may be a hydrogen atom or a methyl group. Even more particularly, Re may be a hydrogen atom.

According to any embodiments of the invention, R^{a} and R^{c}, when taken together, may be a C₃₋₁₀ alkanediyl group optionally comprising one or two functional groups selected amongst ether, ester, carbonyl, amine, amide or alcohol group. Particularly, R^{a} and R^{c}, when taken together, may be a C₃₋₈ alkanediyl group optionally comprising one or two functional groups selected amongst ether, ester, carbonyl, amine, amide or alcohol group. Particularly, R^{a} and Re, when taken together, may be a C₃₋₆ alkanediyl group. Particularly, R^{a} and R^{c}, when taken together, may be a C₄₋₆ alkanediyl group. Even more particularly, R^{a} and R^{c}, when taken together, may be a C₅₋₆ alkanediyl group.

According to any embodiments of the invention, the α-amino carboxylate has from 4 to 10 carbon atoms.

According to any embodiments of the invention, the α-amino carboxylate may be different or identic.

According to any embodiments of the invention, the α-amino carboxylate may be derived from a α-amino acid of natural or artificial origin and may be selected from the group of natural α-amino acid, such as S-alanine or R-alanine (R^{a}=CH₃; R^{b} and R^{c}=H), S-asparagine or R-asparagine (R^{a}=CH₂CONH₂; R^{b} and R^{c}=H), S-glutamine or R-glutamine [R^{a}=(CH₂)₂CONH₂; R^{b} and R^{c}=H], S-isoleucine or R-isoleucine [R^{a}=C(CH₃)CH₂CH₃; R^{b} and R^{c}=H], S-leucine or R-leucine [R^{a}=CH₂CH(CH₃)₂; R^{b} and R^{c}=H], S-lysine or R-lysine [R^{a}=(CH₂)₄NH₂; R^{b} and R^{c}=H], S-methionine or R-methionine [R^{a}=(CH₂)₂SCH₃; R^{b} and R^{c}=H], S-phenylalanine or R-phenylalanine (R^{a}=CH₂C₆H₅; R^{b} and R^{c}=H), S-serine or R-serine (R^{a}=CH₂OH, R^{b} and R^{c}=H), S-tyrosine or R-tyrosine (R^{a}=CH₂C₆H₄OH, R^{b} and R^{c}=H), S-proline or R-proline (R^{a} and R^{c} are taken together=CH₂CH₂CH₂; R^{b}=H); S-valine or R-valine [R^{a}=CH(CH₃)₂; R^{b} and R^{c}=H], S-aspartic acid or R-aspartic acid (R^{a}=CH₂COOH, R^{b} and R^{c}=H), and S-glutamic acid or R-glutamic acid [R^{a}=(CH₂)₂COOH, R^{b} and R^{c}=H], or of an artificial α-amino acid selected from the group of norleucine [R^{a}=(CH₂)₃CH₃; R^{b} and R^{c}=H], norvaline [R^{a}=(CH₂)₂CH₃; R^{b} and R^{c}=H], 2-phenylglycine (R^{a}=C₆H₅; R^{b} and R^{c}=H), ornithine [R^{a}=(CH₂)₃NH; R^{b} and R^{c}=H₂], homoalanine (R^{a}=CH₂CH₃; R^{b} and R^{c}=H), 2-amino-2-methylpropanoic acid R^{a} and R^{b}=CH₃; R^{c}=H) and homoserine [R^{a}=(CH₂)₂OH, R^{b} and R^{c}=H]. Particularly, the α-amino carboxylate may be derived from a α-amino acid may selected from the group consisting of proline, valine, lysine, 2-phenylglycine, phenyl alanine, 2-amino-2-methylpropanoic acid and a mixture thereof.

According to any embodiments of the invention, the β-amino carboxylate is anthralinate.

According to any embodiments of the invention, the amino phenol is selected from the group consisting of 2-aminophenol, 2-aminomethylphenol, 2-Amino-4-chlorophenol, 2-Amino-3-methylphenol, 2-Amino-5-chlorophenol, 2-Amino-4-nitrophenol, 2-Amino-5-nitrophenol, 2-Amino-3-nitrophenol, 2-Amino-4-methoxyphenol, 2-Amino-4-tertbutylphenol and a mixture thereof.

Alternatively, the invention's process for the rearrangement of an epoxide into allylic alcohol may be performed in the presence of a catalyst of formula Zn(carboxylate)₂. So another object of the invention is a process for the rearrangement of an epoxide into an allylic alcohol wherein said process is carried out in the presence of
i) a catalyst of formula Zn(carboxylate)₂; and
ii) an amino phenol;
   wherein the mole ratio between the catalyst of formula Zn(carboxylate)₂ and the amino phenol is comprised between 1:3 and 1:5.

The term “carboxylate” is understood as ligand comprising a COO functional group. Particularly, the carboxylate is of formula R^{d}COO wherein R^{d} is a C₁₋₁₈ alkyl group or a C₆-aryl group, particularly a C₁₋₁₆ alkyl group. The carboxylate may be selected from the group consisting of acetate, octoate, 2-ethyl-hexanoate, laurate, palmitate, stearate, 2-octyldodecanoate, benzoate, naphthenate.

The term “aryl group” designates the normal meaning in the art; i.e. an aromatic hydrocarbon group such as phenyl or naphthyl group optionally substituted. Non-limiting examples of the optional substituent of the aryl group may include C₁₋₃ alkyl or alkoxy group, a hydroxy group or a halogen atom.

According to any one of the invention's embodiments, the catalyst of formula Zn(carboxylate)₂ and the amino phenol is comprised between 1:3 and 1:4.

According to any one of the invention's embodiments, the epoxide, the allylic alcohol and the amino phenol have the same meaning as defined above.

The catalyst of formula Zn(aa)₂ or of formula Zn(carboxylate)₂ can be added into the reaction medium of the invention's process to form an allylic alcohol in a large range of concentrations. As non-limiting examples, one can cite, as catalyst concentration values those ranging from 0.1 mol % to 10 mol %, relative to the total amount of the epoxide. Particularly, the catalyst concentration may be comprised between 0.25 mol % to 5 mol %. It goes without saying that the process works also with more catalyst. However the optimum concentration of catalyst will depend, as the person skilled in the art knows, on the nature of the latter, on the nature of the epoxide, on the temperature and on the desired time of reaction.

The catalyst of formula Zn(aa)₂ is commercially available compound or can be prepared by several methods, such as the one reported in Polymers (Basel, Switzerland) (2019), 11 (5), 790. Alternatively, the catalyst of formula Zn(aa)₂ is formed in situ by the reaction between ZnO and 2 equivalents of AA.

The catalyst of formula Zn(carboxylate)₂ is commercially available compound or can be prepared by several methods, such as the one reported in Spectrochimica Acta, Part A: Molecular and Biomolecular Spectroscopy (2008), 70A(1), 217. Alternatively, the catalyst of formula Zn(carboxylate)₂ is formed in situ by the reaction between ZnO and 2 equivalents of carboxylique acid.

The amino phenol can be added into the reaction medium of the invention's process to form an allylic alcohol in a large range of concentrations. As non-limiting examples, one can cite, as amino phenol concentration values those ranging 1 mol % to 5 mol %, or even between 1 mol % to 3 mol %, relative to the amount of the epoxide. It goes without saying that the process works also with more amino phenol. However, the optimum concentration of amino phenol will depend, as the person skilled in the art knows, on the nature of the latter, on the nature of the epoxide, on the nature of the catalyst, on the temperature and on the desired time of reaction.

According to any one of the invention's embodiments, the invention's process for the rearrangement of an epoxide into allylic alcohol is carried out at a temperature comprised between 160° C. and 190° C. In particular, the temperature is in the range between 170° C. and 190° C. Of course, a person skilled in the art is also able to select the preferred temperature as a function of the melting and boiling point of the starting and final products as well as the desired time of reaction or conversion.

The invention's process for the rearrangement of an epoxide into allylic alcohol can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene or pseudocumene, or mixtures thereof, hydrocarbon solvents such as limonene, decane, dodecane or heavier or mixtures thereof. The choice of the solvent is function of the nature of the substrate and/or catalyst and the person skilled in the art is well able to select the solvent most suitable in each case to optimize the reaction.

The invention's process for rearrangement of an epoxide into allylic alcohol may carried out under batch or continuous conditions.

The invention's process for the rearrangement of an epoxide into allylic alcohol may be performed under atmospheric pressure.

According to any embodiments of the invention, the invention's process further comprises the step of oxidizing the allylic alcohols obtained following the process as defined above into alpha, beta-unsaturated carbonyl compounds.

According to any embodiments of the invention, the alpha,beta-unsaturated carbonyl compound is a compound of formula

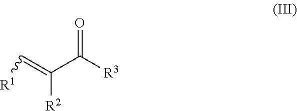

in a form of any one of its stereoisomers or a mixture thereof and wherein R¹, R² and R³ have the same meaning as defined above.

According to any embodiments of the invention, the alpha, beta-unsaturated carbonyl compound is carvone.

According to any embodiments of the invention, the oxidation is carried out in the presence of a Zirconium catalyst and a hydrogen acceptor.

According to any embodiments of the invention, the Zirconium catalyst is of formula

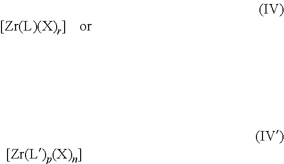

wherein L is a bisphenolate a triphenolate or a calixarene with at least 4 phenol units, L′ is a phenolate, X is an anionic ligand, p is 1 when n is 3 or p is 2 when n is 2 or p is 0 when n is 4 and r is 2 when L is a bisphenolate or r is 1 when L is a triphenolate or r is 0 when L is a calixarene with at least 4 phenol units.

According to any embodiment of the invention, the phenolate is selected from the group consisting of 2-nitrophenolate, ortho-cresolate, 2-(trifluoromethyl) phenolate and 2-cyanophenolate.

According to any embodiment of the invention, the bisphenolate may be selected, but are not limited, from the group consisting of [1,1′-biphenyl]-2,2′-diol, [1,1′-binaphthalene]-2,2′-diol, 6,6′-methylenebis(2,4-di-tert-butylphenol), 6,6′-(ethane-1,1-diyl)bis(2,4-di-tert-butylphenol), 6,6′-methylenebis(2-(tert-butyl)-4-methylphenol), 6,6′-oxybis(2-(tert-butyl)-4-methylphenol) and 6,6′-thiobis(2-(tert-butyl)-4-methylphenol). According to any embodiment of the invention, the anionic ligand, independently from each other, may be halogen atom, a β-diketonate, a OOCR⁷ group or a OR⁸ group wherein R⁷ is a C₁₋₁₀ alkyl group, a benzyl group, a naphtyl group or a phenyl group optionally substituted by a hydroxy group and R⁸ is a C₁₋₆ alkyl group. The term “β-diketonate” is understood as a ligand comprising a C(═O)—CH═C(O⁻) group. Particularly, the β-diketonate is of formula R⁹—C(═O)—CH═C(O⁻)—R¹⁰ wherein R⁹ and R¹⁰, independently from each other, are a C₁₋₆ alkyl group, particularly a C₁₋₄ alkyl group, even more particularly a methyl, propyl, isopropyl or a tertbutyl group. Non-limiting example of β-diketonate may include 4-oxopent-2-en-2-olate, 2,2-dimethyl-5-oxohex-3-en-3-olate, 2,6-dimethyl-5-oxohept-3-en-3-olate or 2,2,6,6-tetramethyl-5-oxohept-3-en-3-olate. Particularly, R⁷ may be C₁₋₈ alkyl group, particularly a C₁₋₆ alkyl group, even more particularly a C₁₋₅ alkyl group. Particularly, R⁸ may be C₁₋₄ alkyl group, particularly a propyl group, a isopropyl group, a butyl group or a tertbutyl group. Particularly, the anionic ligand is selected from the group consisting of acetylacetonate.

According to a particular embodiment, when p is 0 and n is 4; X is a alkodie of formula OR⁹ as define above.

According to any embodiment of the invention, the catalyst of formula (IV) may be selected from the group consisting of Zirconium, [2,2′-methylenebis[(4-methyl-6-tert-butyl)phenolato]]bis(2,4-pentanedionato); Zirconium, [2,2′-methylenebis[(4-6-di-tert-Zirconium, [2,2′-methylenebis[(4-6-di-butyl)phenolato]]bis(2,4-pentanedionato); methyl)phenolato]]bis(2,4-pentanedionato); Zirconium, [2,2′-ethylidenenebis[(4-6-di-tert-butyl)phenolato]]bis(2,4-pentanedionato); Zirconium, [2,2′-thiobis[(4-methyl-6-tert-butyl)phenolato]]bis(2,4-pentanedionato); Zirconium, [2,2′-oxobis[(4-methyl-6-tert-butyl)phenolato]]bis(2,4-pentanedionato); Zirconium, [[2,2′-binaphthalene]-1,1′-diolato]bis(2,4-pentanedionato); Zirconium, [[2,2′-binaphthalene]-1,1′-diolato]bis(propanolate); Zirconium, [[2,2′-biphenyl]-1,1′-diolato]bis(propanolate).

The catalyst of formula (IV) or (IV′) can be added into the reaction medium of the invention's process to form an alpha, beta-unsaturated carbonyl compound in a large range of concentrations. As non-limiting examples, one can cite, as catalyst concentration values those ranging from 0.1 mol % to 10 mol %, relative to the total amount of the allylic alcohol. Particularly, the catalyst concentration may be comprised between 0.5 mol % to 5 mol %. It goes without saying that the process works also with more catalyst. However the optimum concentration of catalyst will depend, as the person skilled in the art knows, on the nature of the latter, on the nature of the allylic alcohol, on the temperature and on the desired time of reaction.

According to any embodiment of the invention, the hydrogen acceptor is selected from the group consisting of benzaldehyde, cyclohexanone, 2-heptanone, 2-octanone, 2-pentanone, acetophenone, 4-methyl-2-pentanone, isophorone, 3-methyl-2-butanone and a mixture thereof.

The hydrogen acceptor can be added into the reaction medium of the invention's process to form an alpha,beta-unsaturated carbonyl compound in a large range of concentrations. As non-limiting examples, one can cite, as hydrogen acceptor concentration values those ranging 1 equivalent to 5 equivalents, or even between 1 equivalent to 2 equivalents, relative to the amount of the allylic alcohol. It goes without saying that the process works also with more hydrogen acceptor. However the optimum concentration of hydrogen acceptor will depend, as the person skilled in the art knows, on the nature of the latter, on the nature of the allylic alcohol, on the temperature and on the desired time of reaction.

According to any one of the invention's embodiments, the invention's process for the oxidation of an allylic alcohol into an alpha,beta-unsaturated carbonyl compound is carried out at a temperature comprised between 50° C. and 190° C. In particular, the temperature is in the range between 100° C. and 140° C. Of course, a person skilled in the art is also able to select the preferred temperature as a function of the melting and boiling point of the starting and final products as well as the desired time of reaction or conversion.

The invention's process for the oxidation of an allylic alcohol into an alpha, beta-unsaturated carbonyl compound can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene or pseudocumene, or mixtures thereof, hydrocarbon solvents such as cyclohexane, heptane or mixtures thereof or mixtures thereof. The choice of the solvent is function of the nature of the substrate and/or catalyst and the person skilled in the art is well able to select the solvent most suitable in each case to optimize the reaction.

The invention's process for the oxidation of an allylic alcohol into an alpha,beta-unsaturated carbonyl compound may be carried out under batch or continuous conditions.

The invention's process for the oxidation of allylic alcohol into alpha, beta-unsaturated carbonyl compound may be performed under atmospheric pressure.

According to a particular embodiment of the invention, the rearrangement of the epoxide into allylic alcohol and the oxidation of the allylic alcohol into alpha,beta-unsaturated carbonyl compound can be performed in one-pot. The term “one-pot” is understood as both steps are performed successively in a single reaction system. In this case, the hydrogen acceptor and the zirconium catalyst are added after completion of the 1^{st} step. The Zirconium catalyst may also be generated in situ after completion of the 1^{st} step.

Another object of the present invention is a catalytic system comprising or consisting of
i) a catalyst of formula Zn(aa)₂ wherein aa is an α-amino acid carboxylate having at least 3 carbon atoms or a β-amino carboxylate; and
ii) an amino phenol.

According to any embodiment of the invention, the catalytic system as defined above is suitable for use in the process for rearrangement of epoxides into allylic alcohols as defined above.

Another object of the present invention is the use of a catalytic system comprising or consisting of
i) a catalyst of formula Zn(aa)₂ wherein aa is an α-amino acid carboxylate having at least 3 carbon atoms or a β-amino carboxylate; and
ii) an amino phenol.
   in the process for the rearrangement of epoxides into allylic alcohols.

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). The preparation of precatalysts and ligands solutions were carried out under an inert atmosphere (Argon) using standard Schlenk techniques. The solvents were dried by conventional procedures and distilled under an argon atmosphere. The NMR of all prepared compounds are in accordance with NMR reported in the literature.

### Example 1

Rearrangement of 1,2-limonene Oxide into Carveol Using Invention's Conditions and Comparative Conditions

A mixture of 1,2-limonene oxide (60% cis isomer and 40% trans isomer), the Zn-catalyst and the 2-amino phenol (2-AP) (equivalents ratio AP/Zn are specified in table I), was heated up to 185° C. in a glass reactor equipped with a stirrer, a thermometer, a Dean-Stark trap, and a condenser. Internal temperature was kept at 185° C. during the entire reaction duration. Crude product was then flash distilled in order to determine the quantity of residues formed during the reaction and yield was calculated based on GC purity of distilled product. Conversion, selectivity (sum of Carveol and Carvone/Conversion) and isolated yields are reported in table 1.

TABLE 1
Rearrangement of 1,2-limonene oxide into carveol using
invention's conditions and comparative conditions

| | | | | | | |
|---|---|---|---|---|---|---|
| | Catalyst | 2-AP/Zn | Time | Conversion | Selectivity | Isolated |
| Catalyst | [mol. %] | [eq.] | [h] | [GC %] | [GC %] | yield [%] |

Invention's Conditions

| | | | | | | |
|---|---|---|---|---|---|---|
| [Zn(2-Ethylhexanoate)₂] | 0.50% | 3 | 12 | 97% | 89.8% | 82.5% |
| [Zn(2-Ethylhexanoate)₂] | 1% | 3 | 12 | 98% | 88.1% | 83.1% |
| [Zn(L-Phengly)₂] | 0.50% | 3 | 20 | 97.1% | 88.7% | 81.6% |
| [Zn(D-Phengly)₂] | 1% | 1.5 | 20 | 97.1% | 88.7% | 80.1% |
| [Zn(D-Phengly)₂] | 1% | 3 | 15 | 95% | 89.4% | 84.5% |
| [Zn(L-valine)₂] | 0.50% | 3 | 16 | 97.2% | 88.4% | 80.0% |
| [Zn(Amino ibutyrate)₂] | 0.50% | 3 | 15 | 96.5% | 86.9% | 79.8% |
| [Zn(L-Phenylalanine)₂] | 0.50% | 3 | 15 | 97.9% | 84.9% | 78.1% |
| [Zn(L-Proline)₂] | 0.50% | 3 | 9 | 98.0% | 88.9% | 82.2% |
| [Zn(L-Proline)₂] | 1% | 1.5 | 7 | 98.9% | 87.8% | 82.4% |
| [Zn(L-Proline)₂] | 1% | 3 | 7 | 99.1% | 88.4% | 83.4% |
| [Zn(D-Proline)₂] | 1% | 1.5 | 7 | 98.3% | 87.8% | 81.8% |
| [Zn(L-Lisine)₂] | 0.50% | 3 | 21 | 97.8% | 87.5% | 80.6% |
| [Zn(2-EH)₂] | 0.25% | 4 | 16 | 99.4% | 89.8% | 85.3% |
| [Zn(L-Proline)₂] | 0.25% | 4 | 9 | 99.0% | 89.0% | 85.5% |

Comparative examples

| | | | | | | |
|---|---|---|---|---|---|---|
| [Zn(2-2-Ethylhexanoate)₂] | 1% | 1.5^{a)} | 12 | 57.0% | 91.3% | |
| [Zn(2-2-Ethylhexanoate)₂]^{b)} | 1% | 1.5 | 3 | 98.0% | 80% | 74% |
| [Zn(glycinate)₂ H₂O] | 0.5% | 1.5 | 7 | 0.3% | — | — |
| [Zn(glycinate)₂ H₂O] | 0.5% | 3 | 7 | 0.4% | — | — |
| [Zn(glycinate)₂ H₂O]^{b)} | 0.5% | 1.5 | 2 | 37.6% | 98% | — |

^{a)}2-AP/Zn ratio as described in WO03/004448 and in WO 2021151790.
^{b)}The reaction was run at 200° C. instead of 185° C.

### Example 2

Preparation of R-(−)-carvone Via One-Pot Successive Rearrangement of R-(+)-1,2-limonene Oxide Using Zn(L-Prolinate)₂ as a Catalyst and Oppenauer Oxidation of R-(−)-carveol in the Presence of Zirconium, [2,2′-methylenebis[(4-methyl-6-tert-butyl)phenolato]]bis(2,4-pentanedionato) and Cyclohexanone as Hydrogen Acceptor

A mixture of R-(+)-1,2-limonene oxide (R-LMO), Zn(L-Prolinate)₂ (0.25 mol. %) and 2-aminophenol (4 eq./Zn) was heated at 185° C. for 9 hours in a glass reactor equipped with a stirrer, a thermometer, a Dean-Stark trap, and a condenser. When the rearrangement step was completed, the reaction mixture was cooled down to room temperature. 1 mol. % of Zirconium, [2,2′-methylenebis[(4-methyl-6-tert-butyl)phenolato]]bis(2,4-pentanedionato) and 1.1 equivalent of cyclohexanone compared to initial R-LMO were added. The reaction mixture was heated at 130° C. for 4 hours to complete conversion of carveol into carvone. The distillation afforded carvone in 99% yields based on intermediate carveol title and 82% based on initial R-LMO. This material was purified using conventional methods to obtain R-(−)-carvone in >99 GC %.

### Example 3

Preparation of R-(−)-carvone Via One-Pot Successive Rearrangement of R-(+)-1,2-limonene Oxide Using Zn(L-Prolinate)₂ as a Catalyst and Oppenauer Oxidation of R-(−)-carveol in the Presence of Zirconium, [2,2′-methylenebis[(4-methyl-6-tert-butyl)phenolato]]bis(2,4-pentanedionato) and Benzaldehyde as Hydrogen Acceptor

A mixture of R-(+)-1,2-limonene oxide (R-LMO), Zn(L-Prolinate)₂ (0.25 mol. %) and 2-aminophenol (4 eq./Zn) was heated at 185° C. for 9 hours in a glass reactor equipped with a stirrer, a thermometer, a Dean-Stark trap, and a condenser. When the rearrangement step was completed, the reaction mixture was cooled down to room temperature. 1 mol. % of Zirconium, [2,2′-methylenebis[(4-methyl-6-tert-butyl)phenolato]]bis(2,4-pentanedionato) and 1.1 equivalent of benzaldehyde compared to initial R-LMO were added. The reaction mixture was heated at 130° C. for 4 hours to complete conversion of carveol into carvone. The distillation afforded carvone in 96% yields based on intermediate carveol title and 79% based on initial R-LMO. This material was purified using conventional methods to obtain R-(−)-carvone in >99 GC % purity.

### Example 4

Preparation of R-(−)-carvone Via One-Pot Successive Rearrangement of R-(+)-1,2-limonene Oxide Using Zn(L-Prolinate)₂ as a Catalyst and Oppenauer Oxidation of R-(−)-carveol in the Presence of Zirconium, [2,2′-methylenebis[(4-methyl-6-tert-butyl)phenolato]]bis(2,4-pentanedionato) and Benzaldehyde as Hydrogen Acceptor

A mixture of R-(+)-1,2-limonene oxide (R-LMO), Zn(L-Prolinate)₂ (0.25 mol. %) and 2-aminophenol (4 eq./Zn) was heated at 185° C. for 9 hours in a glass reactor equipped with a stirrer, a thermometer, a Dean-Stark trap, and a condenser. When the rearrangement step was completed, the reaction mixture was cooled down to room temperature. 1 mol. % of Zirconium, [2,2′-methylenebis[(4-methyl-6-tert-butyl)phenolato]]bis(2,4-pentanedionato) and 1.1 equivalent of benzaldehyde compared to initial R-LMO were added. The reaction mixture was heated at 100° C. for 2 hours then 130° C. for further 2 hours to complete conversion of carveol into carvone. The distillation afforded carvone in 99% yields based on intermediate carveol title and 82% based on initial R-LMO. This material was purified using conventional methods to obtain R-(−)-carvone in >99 GC % purity.

### Example 5

Preparation of R-(−)-carvone Via One-Pot Successive Rearrangement of R-(+)-1,2-limonene Oxide Using Zn(2-ethylhexanoate)₂ as a Catalyst and Oppenauer Oxidation of R-(−)-carveol in the Presence of Zirconium, [2,2′-methylenebis[(4-methyl-6-tert-butyl)phenolato]]bis(2,4-pentanedionato) and Benzaldehyde as Hydrogen Acceptor

A mixture of R-(+)-1,2-limonene oxide (R-LMO), Zn(2-Ethylhexanoate)₂ (0.5 mol. %) and 2-aminophenol (3 eq./Zn) was heated at 185° C. for 15 hours in a glass reactor equipped with a stirrer, a thermometer, a Dean-Stark trap, and a condenser. When the rearrangement step was completed, the reaction mixture was cooled down to room temperature. 1 mol. % of Zirconium, [2,2′-methylenebis[(4-methyl-6-tert-butyl)phenolato]]bis(2,4-pentanedionato) and 1.1 equivalent of benzaldehyde compared to initial R-LMO were added. The reaction mixture was heated at 100° C. for 2 hours then 130° C. for further 2 hours to complete conversion of carveol into carvone. The distillation afforded carvone in 99% yields based on intermediate carveol title and 83% based on initial R-LMO. This material was purified using conventional methods to obtain R-(−)-carvone in >99 GC % purity.

### Example 6

Preparation of R-(−)-carvone Via One-Pot Successive Rearrangement of R-(+)-1,2-limonene Oxide Using Zn(2-ethylhexanoate)₂ as a Catalyst and Oppenauer Oxidation of R-(−)-carveol in the Presence of Zirconium, [2,2′-methylenebis[(4-methyl-6-tert-butyl)phenolato]]bis(2,4-pentanedionato) Generated In Situ and Benzaldehyde as Hydrogen Acceptor

A mixture of R-(+)-1,2-limonene oxide (R-LMO), Zn(2-Ethylhexanoate)₂ (0.5 mol. %) and 2-aminophenol (3 eq./Zn) was heated at 185° C. for 15 hours in a glass reactor equipped with a stirrer, a thermometer, a Dean-Stark trap, and a condenser. When the rearrangement step was completed, the reaction mixture was cooled down to 30° C. 1 mol. % of Zirconium (propoxide) 4 (70% in propanol), 6,6′-methylenebis(2-(tert-butyl)-4-methylphenol) and 2.2 eq. of acetylacetone were added and the reaction mixture was stirred for 1h. 1.1 equivalent of benzaldehyde compared to initial R-LMO were then added. The reaction mixture was heated at 100° C. for 2 hours then 130° C. for further 2 hours to complete conversion of carveol into carvone. The distillation afforded carvone in 99% yields based on intermediate carveol title and 83% based on initial R-LMO. After rectification, the excess of benzaldehyde and co-produced benzyl alcohol were recovered separately in 95% overall yield and >99% purity while R-(−)-carvone was obtained in 98% yield and >99 GC % purity.

### Example 7

Rearrangement of 3,8,8-trimethyl-4-oxatricyclo[5.1.0.0^{3,5}]octane into 4,7,7-trimethylbicyclo[4.1.0]hept-4-en-3-ol Using Invention's Conditions

A mixture of 3,8,8-trimethyl-4-oxatricyclo[5.1.0.0^{3,5}]octane (5 g, 32 mmol), Zn(L-Prolinate) 2 (0.25 mol. %) and 2-aminophenol (4 eq./Zn) was heated at 185° C. for 5 hours in a Schlenk equipped with a magnetic stirrer, a thermometer and a condenser to complete conversion. After distillation on residues the product was obtained in 85% yield as a 70:30 mixture of 4,7,7-trimethylbicyclo[4.1.0]hept-4-en-3-ol and 5-isopropenyl-2-methyl-cyclohex-3-en-1-ol.

### Example 8

Rearrangement of α-Pinene oxide into 6,6-dimethyl-2-methylenebicyclo[3.1.1]heptan-3-ol Using Invention's Conditions

A mixture of α-Pinene oxide (5 g, 32 mmol), Zn(L-Prolinate)₂ (1 mol. %) and 2-aminophenol (3 eq./Zn) was heated at 190° C. for 7 hours in a Schlenk equipped with a magnetic stirrer, a thermometer and a condenser to 80 GC % conversion. 2-methylenebicyclo[3.1.1]heptan-3-ol was obtained in 75% GC selectivity.

### Example 9

Rearrangement of β-Pinene oxide epoxide into (6,6-dimethylbicyclo[3.1.1]hept-2-en-2-yl)methanol Using Invention's Conditions

A mixture of Zn(Phenylglycinate)₂ (60 mg, 0.16 mmol) and 2-aminophenol (4 eq./Zn) in 1.3-diisopropylbenzene (2 mL) was heated for 1 h at 180° C. in a Schlenk equipped with a magnetic stirrer, a thermometer and a condenser. Then 8-Pinene oxide (5 g, 32 mmol) was slowly added and heated for 6 h to 72 GC % conversion. (6,6-dimethylbicyclo[3.1.1]hept-2-en-2-yl)methanol was obtained in 63% GC selectivity.

### Example 10

Preparation of R-(−)-carvone Via One-Pot Successive Rearrangement of R-(+)-1,2-limonene Oxide and Oppenauer Oxidation of R-(−)-carveol in Invention's Conditions and Comparative Conditions

A mixture of 1,2-limonene oxide (LMO, 60% cis isomer and 40% trans isomer, 400 g), the catalyst and 2-amino phenol (2-AP) (equivalents of 2-AP related to Zn are specified in Table 2), was heated up to the temperature specified in Table 2, in a glass reactor equipped with a stirrer, a thermometer, a Dean-Stark trap, and a condenser. The temperature was maintained till conversion of R-LMO was >96%. After cooling down the oxidation catalyst was added together with the hydrogen acceptor (amount is specified in Table 2). In some examples the hydrogen acceptor is added portion wise after selective removal of the hydrogen acceptor (HA) excess and its corresponding alcohol. To clarify, if 2/2 is reported on the time column, this means that half of the HA total amount was added first and after 2 h heating at the reported temperature, the non-reacted HA together with its corresponding alcohol were removed by distillation, then the operation is repeated; e.g. 2/2/2 means that HA was added in 3 times every 2 hours. Crude Carvone was then purified by distillations to obtain Carvone >98% purity. Yield is reported in the column Carvone isolated yield. Separately or together the HA excess and its corresponding alcohol are distilled too. Their overall yield ((mol HA excess+ mol Alcohol)/mol initial total HA) is also reported when possible.

TABLE 2
Rearrangement of 1,2-limonene oxide into carveol followed by oxide and Oppenauer oxidation
to obtained R-(—)-carvone in one-pot using invention's conditions and comparative conditions

| | | Catalyst | 2-AP | T | Carveol | Carvone | | Catalyst |
|---|---|---|---|---|---|---|---|---|
| Entry | Catalyst | mol. % | (eq/Zn) | (° C.) | GC % | GC % | Catalyst | mol. % |
| 1¹⁾ | Zn(octoate)₂ | 1 | 1.5 | 202- | 70% | 13% | Zn(octoate)₂ | 3 |
| | | | | 227 | | | | |
| 2²⁾ | Zn(stearate)₂ | 0.65 | 5.8 | 202- | 74.5% | 4% | Zn(stearate)₂ | 0.65 |
| | | | | 227 | | | | |
| 3²⁾ | Zn(Naphtenate)₂ | 0.65 | 5.8 | 202- | 70.2% | 7.3% | Zn(Naphtenate)₂ | 0.65 |
| | | | | 227 | | | | |
| 4¹⁾ | Zn(octoate)₂ | 1 | 1.5 | 202- | 71.7% | 13% | Zn(octoate)₂ | 3 |
| | | | | 227 | | | | |
| 5³⁾ | Zn(octoate)₂ | 1 | 1.5 | 202- | 70.4% | 12.7% | Zn(octoate)₂ | 3 |
| | | | | 227 | | | | |
| 6⁴⁾ | Zn(octoate)₂ | 0.25 | 4 | 185 | 83.5% | 5.8% | [RaloxZr(acac)₂]⁵⁾ | 1 |
| 7⁴⁾ | Zn(octoate)₂ | 0.25 | 4 | 185 | 83.2% | 5.4% | [RaloxZr(acac)₂]⁵⁾ | 1 |
| 8⁴⁾ | Zn(proline)₂ | 0.25 | 4 | 185 | 84.4% | 4.7% | [RaloxZr(acac)₂]⁵⁾ | 1 |

| | | | | | | | | HA |
|---|---|---|---|---|---|---|---|---|
| | | | | | | | | exces + |
| | Hydrogen | | | | | | Carvone | alcohol |
| | acceptor | HA | Time | T | Carveol | Carvone | Isolated | isolated |
| Entry | (HA) | eq/LO⁶⁾ | (h) | (° C.) | GC % | GC % | yield | yield |
| 1¹⁾ | Cyclohexanone | 1.5 | 2/2/2 | 190 | 0.9% | 82.0% | 70.0% | 83% |
| 2²⁾ | Cyclohexanone | 1.8 | 2/2 | 190 | 38.4% | 43.8% | | disposed |
| 3²⁾ | Cyclohexanone | 1 | 4 | 190 | 18.1% | 56.1% | | disposed |
| 4¹⁾ | Benzaldehyde | 1.5 | 2/2/2 | 190 | 5.0% | 74.0% | 65.0% | 71% |
| 5³⁾ | Benzaldehyde | 1.5 | 2/2/2 | 130 | 51.5% | 24.1% | | |
| 6⁴⁾ | Cyclohexanone | 1.1 | 2 | 130 | | | 82.0% | 90% |
| 7⁴⁾ | Benzaldehyde | 1.1 | 4 | 100- | 0.4% | 89.0% | 83.0% | 95% |
| | | | | 130 | | | | |
| 8⁴⁾ | Benzaldehyde | 1.1 | 4 | 100- | 0.0% | 91.0% | 82.0% | 97% |
| | | | | 130 | | | | |

1) Conditions as reported in WO2003004448
2) Conditions as reported in WO2021151790
3) Conditions as reported in WO2003004448; ecept the temperature of the oxidation
4) Invention's conditions
5) 2,2′-methylenebis[(4-methyl-6-tert-butyl) phenol]
6) 1,2-limonene oxide

The invention's conditions, contrary to the prior art conditions, allow isolating carvone in high yield while recovering the hydrogen acceptor or the corresponding alcohol very efficiency. When the hydrogen acceptor is benzaldehyde, the reduced produced formed is the benzylic alcohol which is a valuable ingredient which could be used as a perfuming ingredient.

1. A process for the rearrangement of epoxides into allylic alcohols, wherein said process is carried out in the presence of
i) a catalyst of formula Zn(aa)₂ wherein aa is an α-amino carboxylate having at least 3 carbon atoms or a β-amino carboxylate; and
ii) an amino phenol.

2. The process according to claim 1, wherein the α-amino carboxylate is of formula R^{a}R^{b}C(NHR^{c})COO⁻ wherein R^{a} is a C₁₋₁₈ hydrocarbon optionally comprising one or two functional groups selected from the group consisting of ether, ester, carbonyl, amine, amide, alcohol, hydroxy, thioether, and carboxylic acid groups, R^{b} and R^{c}, when taken separately, independently from each other, are a hydrogen atom or a C₁₋₁₈ hydrocarbon optionally comprising one or two functional groups selected from the group consisting of ether, ester, carbonyl, amine, amide, alcohol, hydroxy, thioether, and carboxylic acid groups; or R^{a} and R^{c}, when taken together, represent a C₃₋₁₁ alkanediyl group optionally comprising one or two functional groups selected from the group consisting of ether, ester, carbonyl, amine, amide, and alcohol groups.

3. The process according to claim 2, wherein R^{a} is a C₁₋₈ hydrocarbon group optionally comprising one or two functional groups selected from the group consisting of ether, ester, carbonyl, amine, amide, alcohol, hydroxy, thioether, and carboxylic acid groups, R^{b} is a hydrogen atom or a methyl group, and R^{c} is a hydrogen atom or a C₁₋₈ hydrocarbon group optionally comprising one or two functional groups selected from the group consisting of ether, ester, carbonyl, amine, amide, alcohol, hydroxy, thioether, and carboxylic acid groups; or R^{a} and R^{c}, when taken together, represent a C₃₋₈ alkanediyl group optionally comprising one or two functional groups selected from the group consisting of ether, ester, carbonyl, amine, amide, and alcohol group.

4. The process according to claim 2, wherein R^{a} is a phenyl group, a benzyl group or a C₁₋₈ alkyl group optionally comprising one or two functional groups selected from the group consisting of ether, ester, carbonyl, amine, amide, alcohol, hydroxy, thioether, and carboxylic acid groups, R^{b} is a hydrogen atom, and Re is a hydrogen atom, a phenyl group, a benzyl group or a C₁₋₈ alkyl group optionally comprising one or two functional groups selected from the group consisting of ether, ester, carbonyl, amine, amide, alcohol, hydroxy, thioether, and carboxylic acid groups; or R^{a} and R^{c}, when taken together, represent a C₃₋₈ alkanediyl group optionally comprising one or two functional groups selected from the group consisting of ether, ester, carbonyl, amine, amide, and alcohol group.

5. The process according to claim 1, wherein the α-amino carboxylate is selected from the group consisting of proline, valine, lysine, 2-phenylglycine, phenyl alanine, 2-amino-2-methylpropanoic acid and a mixture thereof and the β-amino carboxylate is anthranilate.

6. The process according to claim 1, wherein the amino phenol is selected from the group consisting of 2-aminophenol, 2-aminomethylphenol, 2-Amino-4-chlorophenol, 2-Amino-3-methylphenol, 2-Amino-5-chlorophenol, 2-Amino-4-nitrophenol, 2-Amino-5-nitrophenol, 2-Amino-3-nitrophenol, 2-Amino-4-methoxyphenol 2-Amino-4-tertbutylphenol and a mixture thereof.

7. A process for the rearrangement of epoxides into allylic alcohols, wherein said process is carried out in the presence of
i) a catalyst of formula Zn(carboxylate)₂; and
ii) an amino phenol;
wherein the mole ratio between the catalyst of formula Zn(carboxylate)₂ and the amino phenol is between 1:3 and 1:5.

8. The process according to claim 7, wherein the carboxylate is selected from the group consisting of acetate, octoate, 2-ethyl-hexanoate, laurate, palmitate, stearate, 2-octyldodecanoate, naphthenate, and benzoate.

9. The process according to claim 1, wherein the rearrangement of epoxides into allylic alcohols is carried out at a temperature comprised between 160° C. and 190° C.

10. The process according to claim 1, wherein the epoxide is 1,2-limonene oxide and the allylic alcohols is carveol.

11. The process according to claim 1, wherein the process further comprises the step of oxidizing the allylic alcohols into alpha, beta-unsaturated carbonyl compounds.

12. The process according to claim 11, wherein the alpha, beta-unsaturated carbonyl compound is carvone.

13. The process according to claim 11, wherein the oxidation is carried out in the presence of a Zirconium catalyst and a hydrogen acceptor.

14. The process according to claim 13, wherein the Zirconium catalyst is of formula: 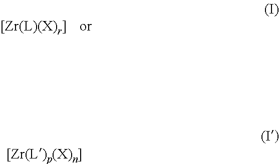
wherein L is a bisphenolate a triphenolate or a calixarene with at least 4 phenol units, L′ is a phenolate, X is an anionic ligand, p is 1 when n is 3 or p is 2 when n is 2 or p is 0 when n is 4 and r is 2 when L is a bisphenolate or r is 1 when L is a triphenolate or r is 0 when L is a calixarene with at least 4 phenol units.

15. The process according to claim 13, wherein the hydrogen acceptor is selected from the group consisting of benzaldehyde, cyclohexanone, 2-heptanone, 2-octanone, 2-pentanone, acetophenone, 4-methyl-2-pentanone, 3-methyl-2-butanone, isophorone, and a mixture thereof.

16. A catalytic system comprising or consisting of:
i) a catalyst of formula Zn(aa)₂ wherein aa is an α-amino acid carboxylate having at least 3 carbon atoms; and
ii) an amino phenol.
